(19) United States
(12) Patent Application Publication
(10) Pub. No.: US 2019/0343768 A1
(43) Pub. Date: Nov. 14, 2019

(54) INSULIN FORMULATIONS FOR RECONSTITUTION INTO HIGH CONCENTRATION LIQUID SOLUTIONS

(71) Applicant: Dance Biopharm Inc., Brisbane, CA (US)

(72) Inventors: Blaine Bueche, San Mateo, CA (US); Matthew Sander, San Francisco, CA (US); Mei-chang Kuo, Palo Alto, CA (US)

(21) Appl. No.: 16/411,757

(22) Filed: May 14, 2019

Related U.S. Application Data
(60) Provisional application No. 62/671,001, filed on May 14, 2018.

(51) Int. Cl. A61K 9/19; A61K 38/28; A61K 9/16; A61K 9/00

(52) U.S. Cl. CPC A61K 9/19; A61K 38/28; A61K 9/1617; A61K 9/1694; A61K 9/0019; A61K 9/0075; A61K 9/0078; A61K 9/0095

(57) ABSTRACT

A dry powder insulin formulation for reconstitution including insulin, a buffering agent, and a salt. The dry powder insulin formulation includes between about 70 and 95% w/w of insulin, between about 5 and 30% w/w of the buffering agent, and less than about 1% w/w of the salt. The dry powder insulin formulation has less than about 5% water content.

### CROSS-REFERENCE TO RELATED APPLICATIONS

This application claims priority to U.S. Provisional Patent Application No. 62/671,001, filed May 14, 2018, the disclosure of which is incorporated by reference herein in its entirety.

### BACKGROUND OF THE INVENTION

Reconstitution of insulin in water is a challenge for dried insulin formulations. Typically, pure microcrystalline insulin is mostly insoluble in water. This is especially true at high concentration. As a result, many commercial manufacturing methods include dissolution of insulin in acids, at low pH, followed by pH adjustments. Pure insulin may be reconstituted as high as 30-75 mg/mL in acidic solution, followed by a pH adjustment. However, this complicated process cannot be repeated by individual patients as patients lack the required instrumentation, such as pH meters, and the training to safely reconstitute formulations to a desired potency.

### BRIEF SUMMARY OF THE INVENTION

The present disclosure relates to systems and methods of a dry powder insulin formulation for reconstitution into a liquid solution for delivery to a patient. Such dry powder formulations may have an especially long, stable shelf life when refrigerated or stored at room temperature and may be reconstituted in pure water at concentrations of greater than 30 mg/mL very quickly with gentle agitation. The reconstitution liquid does not have to be pure water, but can also contain additional buffer salts or components to extend the physical and/or chemical stability of the aqueous solution. The powder formulations may include insulin, a salt, and a buffering agent, and do not include a preservative or stabilizer.

In one aspect, a dry powder insulin formulation for reconstitution is provided. The formulation may include insulin, a buffering agent, and a salt. The dry powder insulin formulation may have less than about 5% water content. In some embodiments, the dry powder insulation formulation may not contain a stabilizer or preservative. In some embodiments, the buffering agent may include one or more of citrate, phosphate, acetate, Tris(hydroxymethyl)aminomethane (TRIS), glycine, or glycylglycine. In some embodiments, the dry powder insulin formulation may be spray-dried, while in others the dry powder insulin formulation may be lyophilized.

In another aspect, a method of reconstituting a dry powder insulin formulation is provided. The method may include providing a dry powder insulin formulation. The dry powder insulin formulation may include insulin, a buffering agent, and a salt. The dry powder insulin formulation may have less than about 5% water content. In some embodiments, the dry powder insulation formulation may not contain a stabilizer or preservative. The method may also include combining the dry powder insulin formulation with a solvent to reconstitute the dry powder insulin formulation into a liquid insulin formulation. The dry powder insulin formulation is substantially dissolved within about 60-120 seconds. Further, the resulting solution is substantially clear. In some embodiments, the liquid insulin formulation may have a concentration of greater than about 35 mg/mL. In some embodiments, the solvent is water.

In another aspect, a method of reconstituting a dry powder insulin formulation is provided. The method may include providing a dry powder insulin formulation in a first chamber of a syringe. The dry powder insulin formulation may include insulin, a buffering agent, and a salt. The dry powder insulin formulation may have less than about 5% water content. In some embodiments, the dry powder insulation formulation may not contain a stabilizer or preservative. The method may also include providing a solvent in a second chamber of the syringe and introducing the solvent into the first chamber to combine the dry powder insulin formulation with the solvent to reconstitute the dry powder insulin formulation into a liquid insulin formulation. The method may further include separating the second chamber from the first chamber and inserting the first chamber into an insulin delivery device. In some embodiments, the delivery device may be a pen injector, an inhaler, or a liquid nebulizer.

In another aspect, a dry powder insulin formulation for reconstitution is provided. The dry powder formulation may consist of insulin, a buffering agent, and a salt. The dry powder insulin formulation may have less than about 5% water content.

In another aspect, a dry powder insulin formulation is provided. The formulation may include insulin, a buffering agent, and a salt. The dry powder insulin formulation may have less than about 5% water content. In some embodiments, the dry powder insulation formulation may not contain a stabilizer or preservative. In some embodiments, the buffering agent may include one or more of citrate, phosphate, acetate, Tris(hydroxymethyl)aminomethane (TRIS), glycine, or glycylglycine. In some embodiments, the dry powder insulin formulation may be spray-dried, while in others the dry powder insulin formulation may be lyophilized.

### BRIEF DESCRIPTION OF THE DRAWINGS

A further understanding of the nature and advantages of various embodiments may be realized by reference to the following figures. In the appended figures, similar components or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

FIG. 1 depicts a flowchart of a process for forming a dry powder insulin formulation according to embodiments.

FIG. 2 depicts a flowchart of a process of reconstituting a dry powder insulin formulation according to embodiments.

FIG. 3A depicts one view of a dual chamber syringe according to embodiments.

FIG. 3B depicts one view of a dual chamber syringe according to embodiments.

FIG. 3C depicts one view of a dual chamber syringe according to embodiments.

FIG. 3D depicts one view of a dual chamber syringe according to embodiments.

FIG. 4 depicts a flowchart of a process of using a dual chamber syringe according to embodiments.

### DETAILED DESCRIPTION OF THE INVENTION

The present disclosure relates to systems and methods of a dry powder insulin formulation for reconstitution into a liquid solution for delivery to a patient. Such dry powder formulations may have an especially long, stable shelf life. For example, the room temperature shelf life of such formulations may be at least about 24 months, and in some cases at least about 60 months. The present formulations are prepared by formulation of a liquid solution, followed by drying the liquid solution into an amorphous powder. For example, the liquid solution may be dried by spray drying or freeze drying. In some embodiments, the amorphous powder preparation may contain a mixture of about 80% insulin and about 20% excipient and is more stable than recombinant human (microcrystalline) insulin.

Present embodiments of dry powder insulin formulations may be reconstituted at high concentrations. For example, concentrations greater than 35 mg/mL (1000 U/mL) may be achieved, which is more than double that of typical insulin formulations. Such high concentrations provide several benefits. High concentration formulations are most suitable for pulmonary delivery, which requires aerosolization using a vibrating mesh nebulizer, an ultrasonic nebulizer, a compressed air (jet) nebulizers, and the like. These devices deliver doses at maximal velocities of approximately 0.5 mL/min. Using such devices, pulmonary doses of 1000 U/mL insulin range from approximately 0.05-0.25 ml, requiring 6-30 seconds of inhalation. Conventional concentrated liquid insulin formulations range in much lower concentration between about 100 U/mL to about 500 U/mL. Typical pulmonary doses of 100 U/mL insulin using the same devices would require 1-10 minutes of inhalation. Such lengthy inhalation times significantly diminish patient compliance and experience. Hence, the formulations of the invention will typically be reconstituted to a concentration of at least about 280 U/mL, and in other cases at least about 2000 U/mL.

The present dry powder formulations have high levels of stability, which provide significant advantages for pharmaceutical manufacturing and storage. These advantages include the ability to store a dry formulation for reconstitution at room temperature for a reasonable shelf life exceeding 2 years. In contrast, pure insulin drug substance cannot be stored at room temperature over a 2 year shelf life.

In some embodiments, a dry powder insulin formulation may be provided that is not designed for reconstitution. Such dry powder formulations may have similar stability as the powders for reconstitution. For example, these dry powder formulations may have a shelf life at room temperature exceeding 2 years. These formulations may include insulin, a buffering agent, and a salt. The dry powder insulin formulations may have less than about 5% water content. In some embodiments, the dry powder insulation formulation may not contain a stabilizer or preservative. In some embodiments, the buffering agent may include one or more of citrate, phosphate, acetate, Tris(hydroxymethyl)aminomethane (TRIS), glycine, or glycylglycine. In some embodiments, the dry powder insulin formulation may be spray-dried, while in others the dry powder insulin formulation may be lyophilized.

Conventionally, reconstituting dry powder insulin formulations is a long process, especially when reconstituting at high concentration levels. Dry powder insulin formulations of present embodiments may be quickly reconstituted in water. Reconstitution of current formulations in pure water can be accomplished at concentrations of greater than 30 mg/mL in approximate 5 minutes with gentle agitation. Some embodiments may provide even quicker reconstitution times, such as under 90 seconds. In contrast, pure insulin cannot be reconstituted to 30 mg/mL in pure water. The ability to reconstitute the formulation to a high concentration in pure water is a significant advantage. For example, such formulations may be reconstituted by patients themselves for use in a variety of delivery methods. In some embodiments, a dual chamber syringe or aerosolizer may be used. One chamber may be filled with the formulation and the other chamber may be filled with a solvent, such as sterile water. The contents of the chambers may be mixed to reconstitute the insulin formulation for delivery of a liquid formulation to the patient.

Additional embodiments may include different reconstitution solvents designed to enhance the liquid formulation stability or properties. For example different buffer systems, alcohols, surfactants, tonicity agents, preservatives and stabilizers could be added.

Increases in environmental robustness can be achieved by increasing the insulin content and by decreasing the percentage of hygroscopic glass-forming excipients. Significant improvements in chemical stability are also noted with decreases in the zinc (Zn) content. Again, this is unexpected, as Zn ions are added to conventional insulin formulations to promote hexamer formation, which has been demonstrated to improve chemical stability relative to monomeric or dimeric insulin. The formulations described herein seek to minimize and/or eliminate hexamer formation by reducing and/or eliminating the zinc content.

In some embodiments, the insulin formulations described herein include insulin, a buffering agent, and a salt. In some embodiments, the insulation formulations may include only insulin, a buffering agent, and a salt. Insulin formulations of the present invention may be dried, such as using spray drying techniques and/or lyophilization, to a water content typically less than 5%. The present insulin formulations have an insulin content in the range of about 70% w/w to 95% w/w, with contents between 85% w/w and 90% w/w, preferred to promote high levels of robustness and chemical stability. The moisture content is typically less than 5% w/w preferred, so as to maintain the glass transition temperature (ca., 45° C. at 5% moisture) significantly higher than room temperature storage conditions. A composition in “dry powder form” is a powder composition that contains less than about 20 wt % moisture, such as less than 10 wt % or less than 5 wt % moisture.

Some embodiments of dry powder formulations may include stabilizing agents and/or preservatives. Examples of stabilizers include, but are not limited to, phenol and derivatives thereof such as meta-cresol, chloro-cresol, methylparaben, ethyl paraben, propyl paraben, thymol, as well as derivatives and mixtures of such compounds. Some similar non-phenol preservatives and stabilizers include, but are not limited to, bi- or tricyclic aliphatic alcohols and purines, such as a bicyclic aliphatic alcohol, including a monoterpenol, such as isopinocampheol, 2,3-pinandiol, myrtanol, bomeol or fenchol, a tricyclic aliphatic alcohol, such as 1-adamantanol, and purines such as adenine, guanine, or hypoxanthine. It will be appreciated that in other embodiments, dry powder insulation formations for reconstitution may be formed without stabilizing agents or preservatives, as these may actually lead to decreased stability when reconstituted.

The insulin formulation described herein may include natural and/or synthetically-derived insulin including analogs thereof. For example, the insulin may include polypeptides having up to two amino acid modifications (deletion, substitution, or addition variants). Such insulin formulations may be produced by any manner including, but not limited to, pancreatic extraction, recombinant expression, and in vitro polypeptide synthesis. Additionally, insulins that are produced by modifying native insulin and compounds that are produced in any manner to provide the desired end product may be included. Thus, it is not necessary to begin with an “unmodified” insulin starting material, such as a native insulin; starting materials for synthesizing the insulin end product may be amino acids. Native insulin refers to human insulin having an amino acid sequence corresponding to the amino acid sequence of human insulin as found in nature. Native insulin can be natural (i.e., isolated from a natural source) or synthetically produced.

Insulin may include any purified isolated polypeptide having part or all of the primary structural conformation (that is to say, contiguous series of amino acid residues) and at least one of the biological properties of naturally occurring insulin. The type of insulin included in the formulations may vary. In some instances, the insulin may be human, porcine, or bovine insulin. In some instances, the formulation includes human insulin. In some instances, the insulin is human insulin. The insulin may be a recombinant protein derived from human or other mammalian cell lines. In some instances, the insulin may be a recombinant insulin protein derived from prokaryotic cells. In some instances, the insulin may be the full-length, 51 amino acid wild-type sequence of the insulin protein. In other instances, the insulin may be an insulin analogue that has a genetically modified sequence. For example, the insulin may have one or more amino acids deleted and/or replaced by other amino acids, including non-codeable amino acids, or may have one or more amino acids added to the protein sequence. In some embodiments, the insulin may include an insulin analog, such as at least one of Lys(B3)-Glu(B29) human insulin; Lys^{B28}Pro^{B29} human insulin, B28 Asp human insulin, human insulin, in which proline in position B28 has been substituted by Asp, Lys, Leu, Val or Ala and where in position B29 Lys can be substituted by Pro; AlaB26 human insulin; des(B28-B30) human insulin; des(B27) human insulin or des(B30) human insulin. In additional embodiments, the polypeptide of the preparation comprises an insulin derivative selected from at least one of B29-N-myristoyl-des(B30) human insulin, B29-N-palmitoyl-des(B30) human insulin, B29-N-myristoyl human insulin, B29-N-palmitoyl human insulin, B28-N-myristoyl Lys^{B28}Pro^{B29} human insulin, B28-N-palmitoyl-Lys^{B28}Pro^{B29} human insulin, B30-N-myristoyl-Thr^{B29}Lys^{B30} human insulin, B30-N-palmitoyl-Thr^{B29}Lys^{B30} human insulin, B29-N-(N-palmitoyl-γ-glutamyl)-des (B30) human insulin, B29-N-(N-lithocholyl-γ-glutamyl)-des(B30) human insulin, B29-N-(ω-carboxyheptadecanoyl)-des(B30) human insulin, and B29-N-(ω-carboxyheptadecanoyl) human insulin. In some embodiments, the insulin may include mixtures of an insulin, an insulin analog, and/or an insulin derivative.

As noted above, oftentimes the dry powder insulin formulations may include a buffering agent and/or a salt. The formulations may also include other excipients, such as carbohydrates, antimicrobial agents, antioxidants, and combinations thereof. Excipients may make up, in total, between about 0% w/w and 40% w/w of the dry powder formulation in some embodiments. Individually, these agents, if present, are generally present in amounts of from about 0.01% to about 10%, by weight, of the composition. In some embodiments, the amount ranges from about 0.02% to about 9%, or from about 0.03% to about 8%, or from about 0.04% to about 7%, or from about 0.05% to about 6% by weight, of the composition. The amount chosen will depend upon its desired effect on the composition and can be varied as needed.

The inventive compositions may further include flavoring agents, taste-masking agents, sweeteners, antistatic agents, surfactants (for example polysorbates such as “TWEEN 20” and “TWEEN 80”), sorbitan esters, lipids (for example phospholipids such as lecithin and other phosphatidylcholines, phosphatidylethanolamines), fatty acids and fatty esters, steroids (for example cholesterol), and/or chelating agents (for example EDTA, zinc and other such suitable cations).

The compositions of the invention may include one or more buffering, or pH-adjusting or -controlling, agents. These agents are generally a salt prepared from an organic acid or base. Representative buffers include organic acid salts of citric acid, ascorbic acid, gluconic acid, carbonic acid, tartaric acid, succinic acid, acetic acid, or phthalic acid, Tris, tromethamine hydrochloride, or phosphate buffers. Suitable amino acids, which may also function in a buffering capacity, include alanine, glycine, arginine, betaine, histidine, glutamic acid, aspartic acid, cysteine, lysine, leucine, isoleucine, valine, methionine, phenylalanine, aspartame, tyrosine, tryptophan, and the like. Other buffering agents usable in these formulations include citrates, phosphates, acetates, Tris(hydroxymethyl)aminomethane (TRIS), glycines, and/or glycylglycines, however other suitable buffering agents may be used as well. Buffering agents may be present in quantities of between about 1 and 10% by mass.

Unfortunately, formulation of these amorphous glass powders with higher insulin content results in decreases in chemical stability due to the reductions in the stabilizing glass-forming excipients. It has been surprisingly discovered that increases in chemical stability can be achieved when insulin inhalation powders are formulated at basic pH. The improvements in stability noted at basic pH are unexpected, as decreases in insulin stability are typically observed in insulin products in this range of pH. Formulation at pH 7.8 has been demonstrated to reduce formation of A21 hydrolysis products, and insulin related compounds including high molecular weight proteins. Therefore, the preferred pH range is greater than 7.5, but less than 9.0, with particularly preferred pHs in the range from 7.6 to 8.5.

Salts may be useful as surface-active germicides for many pathogenic bacteria and fungi and may include, but are not limited to, octadecyldimethylbenzyl ammonium chloride, hexamethonium chloride, benzalkonium chloride (a mixture of alkylbenzyldimethylammonium chloride in which the alkyl groups are long-chain compounds), and benzethonium chloride. Typically, salts, if present at all, are present in quantities of less than 1% by weight.

Pharmaceutically acceptable salts may also include but are not limited to amino acid salts, salts prepared with inorganic acids, such as chloride, sulfate, phosphate, diphosphate, hydrobromide, and nitrate salts, or salts prepared with an organic acid, such as malate, maleate, fumarate, tartrate, succinate, ethylsuccinate, citrate, acetate, lactate, methanesulfonate, benzoate, ascorbate, para-toluenesulfonate, palmitate, salicylate and stearate, as well as estolate, gluceptate, and lactobionate salts. Similarly, salts containing pharmaceutically acceptable cations include, but are not limited to, sodium, potassium, calcium, magnesium, aluminum, lithium, and ammonium (including substituted ammonium). Modified insulins may be in the form of a pharmaceutically acceptable salt.

In some embodiments, a carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like.

The preparation may also include an antimicrobial agent for preventing or deterring microbial growth. Non-limiting examples of antimicrobial agents suitable for the present invention include, but are not limited to, benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the preparation as well. Antioxidants are used to prevent oxidation, thereby preventing the deterioration of the conjugate or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

The insulin formulations described herein may include particles that have physical characteristics that allow for quick reconstituting in water. The dry particles of the present invention may generally have a mass median diameter (MMD), or volume median geometric diameter (VMGD), or mass median envelope diameter (MMED), or a mass median geometric diameter (MMGD), of less than about 2 to 50 μm. Small diameters may be achieved by a combination of optimized spray drying conditions and choice and concentration of excipients. The powders of the present invention may also be characterized by their densities. The powder may possess a bulk density between about 0.1 to 0.5 g/mL.

In view of the present description, the compounds of the present invention may be formulated by various methods and techniques known and available to those skilled in the art. In this regard, the insulin powders of the invention can be formulated in any number of ways. Consequently, the insulin powders provided herein are not limited to the specific technique or approach used in their formulation. Exemplary approaches for formulating the presently described insulin powders, however, will be discussed in detail below.

In one embodiment of the invention, heterogeneous particles are formed by forming a liquid composition comprising insulin and one or more excipients, such as a buffering agent and a salt.

Dry powder formulations may be prepared, for example, by spray drying (or freeze drying or spray-freeze drying). Spray drying of the formulations is carried out, for example, as described generally in the “Spray Drying Handbook”, 5^{th} ed., K. Masters, John Wiley & Sons, Inc., NY, N.Y. (1991), and in WO 97/41833, which are incorporated herein by reference.

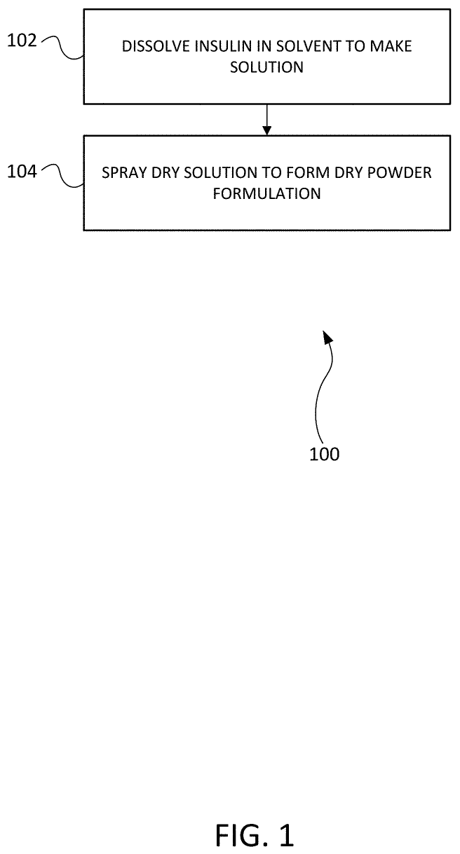

The insulin compositions of the invention can be spray-dried from a solvent, e.g., an aqueous solution. One embodiment of a process 100 for manufacturing the insulin formulations of the present invention is illustrated in FIG. 1. At block 102 insulin is dissolved in water or another solvent. The solvent may contain one or more physiologically acceptable buffering agents, one or more salts, and/or other excipients as described above. The pH range of modified insulin-containing solutions is generally between about 7.5 to 9.0. The aqueous formulation may optionally contain additional water-miscible solvents, such as acetone, alcohols, and the like. Representative alcohols are lower alcohols such as methanol, ethanol, propanol, isopropanol, and the like. The pre-spray dried solutions will generally contain solids dissolved at a concentration from 0.1% to 4%. A dispersibility-enhancing agent, glass-stabilizing agent, and/or precipitating agent may be included in the solution.

At block 104, the solutions are then spray dried in a spray drier, such as those available from commercial suppliers such as Niro A/S (Denmark), Büchi (Switzerland) and the like, resulting in a dispersible, dry powder. Optimal conditions for spray drying the solutions will vary depending upon the formulation components, and are generally determined experimentally. The gas used to spray dry the material is typically air, although inert gases such as nitrogen or argon are also suitable. Moreover, the temperature of both the inlet and outlet of the gas used to dry the sprayed material is such that it does not cause decomposition of the modified insulin in the sprayed material. Such temperatures are typically determined experimentally, although generally, the inlet temperature will range from about 110 to 160° C. while the outlet temperature will range from about 60 to 95° C.

Alternatively, powders may be prepared by lyophilization, vacuum drying, spray-freeze drying, super critical fluid processing, air drying, or other forms of evaporative drying. In some instances, it may be desirable to provide the dry powder formulation in a form that possesses improved handling/processing characteristics, e.g., reduced static, better flowability.

When reconstituted, it is desirable that formulations of such concentrations (e.g. greater than 750 U/mL) have a shelf life at room temperature of at least about 30 days.

Table 1 shows the stability of several embodiments of insulin powder formulations for 9 months at the accelerated condition of 40° C. Stability is measured in terms of the purity of formulated insulin in the dry powders. Ten embodiments are shown. During manufacturing, between about 5% and 50% of the formulation is made up of buffer components and/or pH adjusters, such as NaOH, citric acid, and/or sodium chloride.

TABLE 1
Purity of Insulin Formulations Accelerated
Storage Condition: 40° C.

| | | 1 | 3 | 6 | 9 | % Degrada- |
|---|---|---|---|---|---|---|
| Sample | 0 | month | months | months | months | tion/Month |
| DSD0004 | 99.5 | 99.0 | 98.3 | 97.8 | 96.9 | 0.3 |
| DSD0005 | 99.5 | 99.2 | 98.4 | 98.0 | N/A | 0.2 |
| DSD0006 | 99.5 | 99.2 | 98.3 | 98.2 | 97.4 | 0.2 |
| DSD0007 | 99.4 | 99.2 | 98.7 | 98.3 | 97.5 | 0.2 |
| DSD0008 | 99.4 | 99.0 | 98.5 | 98.0 | 97.1 | 0.2 |
| DSD0009 | 99.4 | 99.1 | 98.6 | 98.2 | 97.6 | 0.2 |
| DSD0010 | 99.3 | 99.1 | 98.5 | 98.0 | 97.2 | 0.2 |
| DSD0011 | 99.4 | 99.3 | 98.7 | 98.4 | 97.6 | 0.2 |
| DSD0012 | 99.4 | 99.4 | 98.7 | 98.4 | 97.6 | 0.2 |
| DSD0013 | 99.4 | 99.3 | 98.8 | 98.4 | 97.8 | 0.2 |
| DSD0014 | 99.4 | 99.3 | 98.9 | 98.4 | 97.7 | 0.2 |

Table 2 shows a percentage of various components in a reconstituted insulin formulation according to some embodiments. In some embodiments, the feedstock solution is around pH 8.0 (oftentimes between about 7.5 and 9.0) with low zinc (under about 0.6% w/w, and in some cases less than about 0.37% w/w) to reduce hexamer population during the freezing or spray drying step this is the key property for enhanced stability of the powder.

TABLE 2
Percent of Composition of Various Components
for Reconstituted Insulin Solutions

| Final | Final | | % Buffer | pH of | % EtOH | % Solids |
|---|---|---|---|---|---|---|
| Batches | % | % | Compo- | Feed- | Feed- | Feed- |
| Powder | Insulin | Water | nents | stock | stock | stock |
| DSD004 | 78 | 6 | 17 | 7.3 | 0.0 | 3.57 |
| DSD005 | 78 | 5 | 17 | 7.3 | 0.0 | 3.57 |
| DSD006 | 78 | 5 | 17 | 7.8 | 0.0 | 3.57 |
| DSD007 | 78 | 5 | 17 | 7.8 | 0.0 | 3.57 |
| DSD008 | 67 | 4 | 29 | 7.8 | 5.0 | 1.79 |
| DSD009 | 68 | 4 | 28 | 7.8 | 5.0 | 1.79 |
| DSD010 | 68 | 4 | 28 | 7.8 | 2.5 | 0.90 |
| DSD011 | 75 | 6 | 20 | 7.8 | 5.0 | ~3.0 |
| DSD012 | 76 | 4 | 20 | 7.8 | 10.0 | ~3.0 |
| DSD013 | 75 | 4 | 20 | 7.8 | 10.0 | ~3.0 |
| DSD014 | 78 | 4 | 18 | 7.8 | 5.0 | ~3.0 |

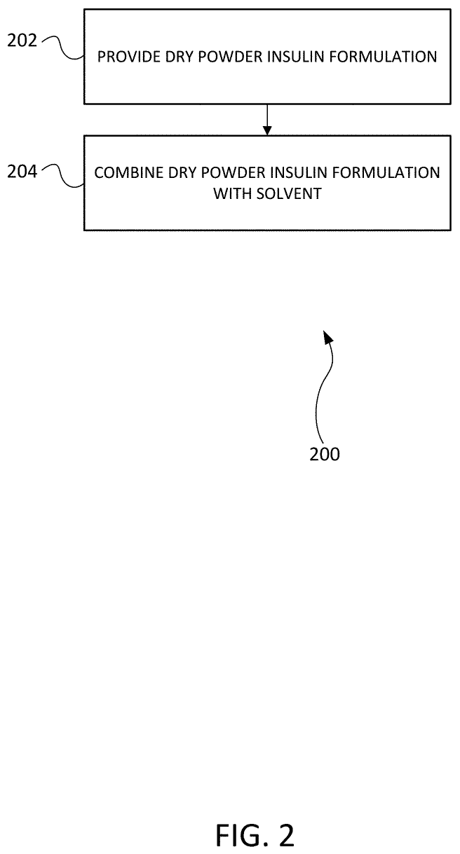

FIG. 2 depicts a process 200 of reconstituting a dry powder insulin formulation as described herein. At block 202, a dry powder insulin formulation is provided. The dry powder formulation may be any of the formulations described herein and may include insulin, a buffering agent, and a salt. The dry powder insulin formulation may have less than about 5% water content and does not contain a stabilizer or preservative. At block 204, the dry powder insulin formulation is combined with a solvent, such as water, to reconstitute the dry powder insulin formulation into a liquid insulin formulation. This reconstitution may be done merely by combining the powder and solvent and/or may include agitating the mixture, such as by shaking or stirring the mixture. The combination may be done in any sterile container suitable for providing the reconstituted liquid insulin formulation to a delivery device, such as an injection pen, inhaler, or nebulizer.

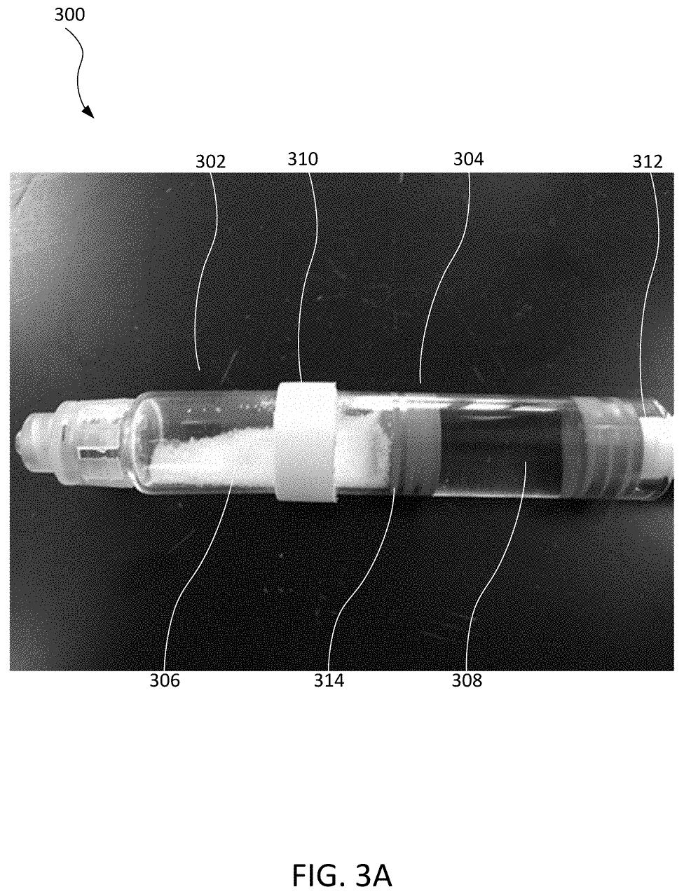

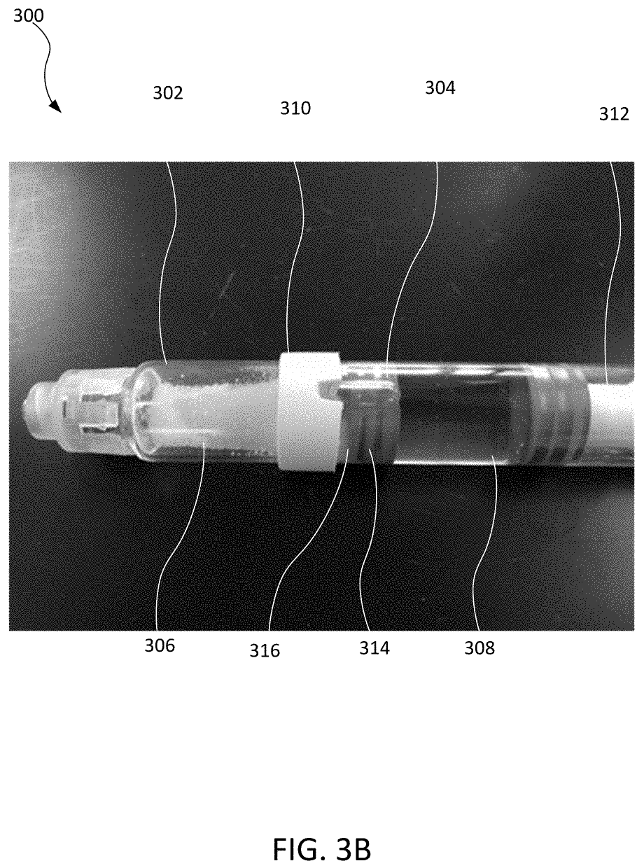

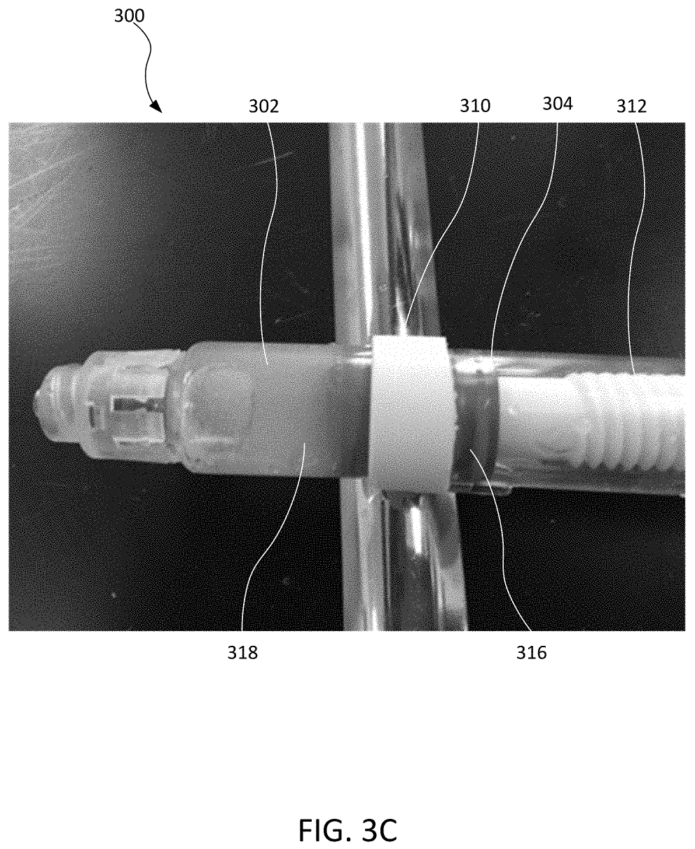

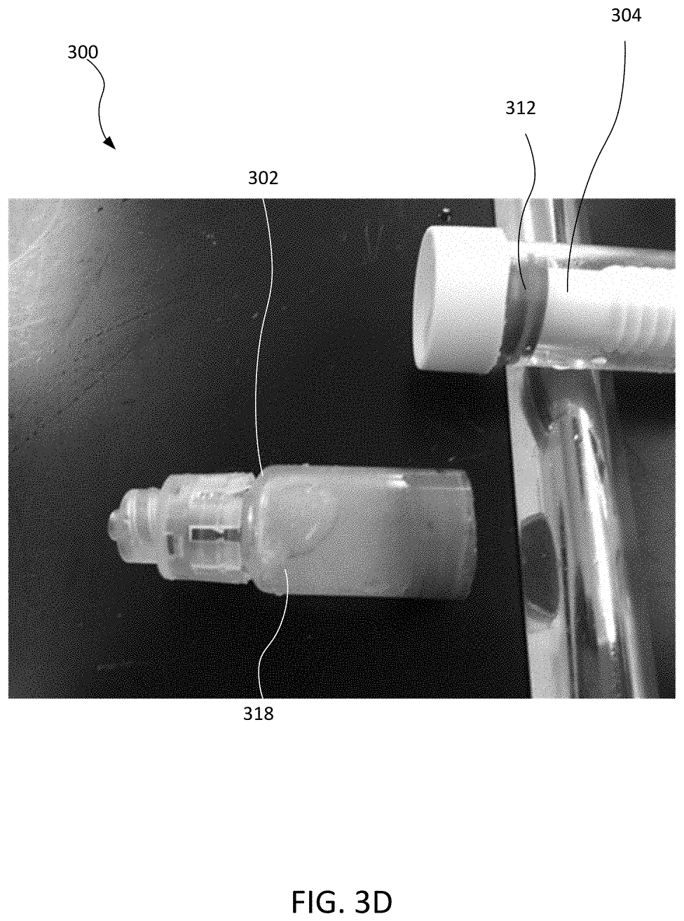

In some embodiments, the formulations described herein may be reconstituted using a dual chamber syringe, such as that shown in FIGS. 3A-3D. FIG. 3A shows one embodiment of a dual chamber syringe 300 having a first chamber 302 and a second chamber 304. Here, first chamber 302 and second chamber 304 are collinear with one another. First chamber 302 may be configured to hold a dry powder insulin formulation 306, while second chamber 304 is configured to hold a solvent 308, such as water. A valve 310 may be positioned between the first chamber 302 and second chamber 304. As shown in FIG. 3B, A plunger 312 may be depressed to force solvent 308 from second chamber 304 through valve 310 and into first chamber 302. In some embodiments, this may be done by the plunger 312 causing a piston 314 to move past valve 310, causing valve 310 to open. Piston 314 may be disposed at a proximal end 316 of second chamber 302 such that by moving piston 314 past valve 310, the solvent 308 is also moved past valve 310 and allowed to enter first chamber 302. In other embodiments, valve 310 may be a check valve 310 or other pressure actuated valve. When plunger 312 is actuated, the solvent 308 is pushed against valve 310 until a pressure against valve 310 reaches a threshold pressure, causing valve 310 to open and allowing solvent 308 to pass through valve 310 into the first chamber 302. Once combined with solvent 308, the dry powder insulin formulation 306 reconstitutes into a liquid insulin formulation 318 as seen in FIG. 3C. The second chamber 304 may be removable, such as once all of the solvent is introduced into the first chamber 302. FIG. 3D shows first chamber 302 separated from second chamber 304. This may be done by unfastening the second chamber 304, such as by unscrewing or unsnapping a fastener from first chamber 302. Upon removal of the second chamber 304, the first chamber 302 containing the liquid insulin may be configured to be inserted into a delivery device, such as a pen injector, inhaler, and/or liquid nebulizer. In other embodiments, the first chamber 302 may be used to store or transfer the liquid insulin to another container for storage, such as for up to 30 days.

In other embodiments, the dried powder insulin formulation may be stored in a bottle or other container. A solvent may be injected into the container for reconstitution. For example, a syringe may contain a solvent, which may be injected into the container using a needle or other delivery mechanism.

While shown as collinearly aligned, it will be appreciated that other designs of dual chamber syringes may be utilized.

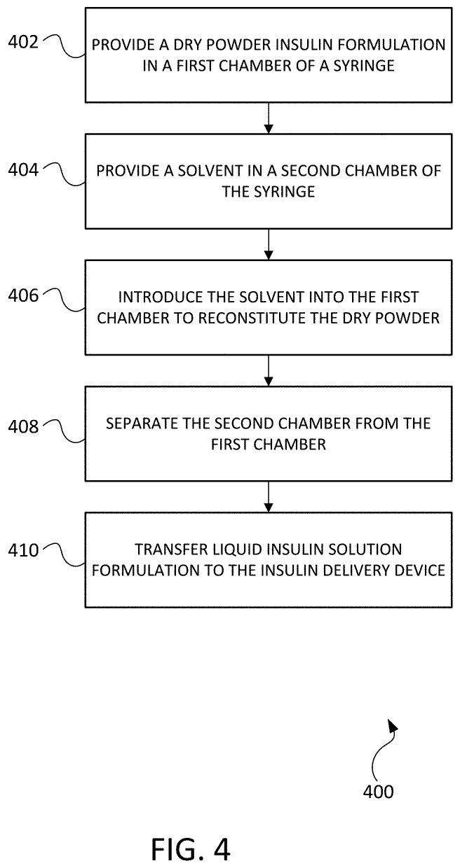

FIG. 4 depicts a method of using a dual chamber syringe, such as dual chamber syringe 300. At block 402, a dry powder insulin formulation is provided in a first chamber of a syringe. The dry insulin powder formulation may be any of the formulations described herein and may include insulin, a buffering agent, and a salt. The buffering agent may include one or more of citrate, phosphate, acetate, Tris(hydroxymethyl)aminomethane (TRIS), glycine, or glycylglycine, among others. The salt may include one or more of chloride, sulfate, phosphate, diphosphate, hydrobromide, and nitrate salts, among others. In some embodiments, the dry powder insulin formulation has less than about 5% water content and does not contain a stabilizer or preservative. At block 404, a solvent is provided in a second chamber of the syringe. In some embodiments, water may be used as the solvent. The solvent is introduced into the first chamber to combine the dry powder insulin formulation with the solvent to reconstitute the dry powder insulin formulation into a liquid insulin formulation at block 406. In some embodiments, the reconstitution may be done within about 60 to 120 seconds. The reconstituted solution may have an insulin concentration between about 30 IU (1/mg/mL) and 2000 IU (70 mg/mL). At block 408, the second chamber if separated from the first chamber The liquid insulin formulation is transferred into an insulin delivery device at block 410, which may involve inserting the first chamber into an insulin delivery device. Suitable delivery devices may include injector pens, inhalers, nebulizers, and the like.

The invention has now been described in detail for purposes of clarity and understanding. However, it will be appreciated that certain changes and modifications may be practiced within the scope of the appended claims.

What is claimed is:

1. A dry powder insulin formulation for reconstitution comprising:
insulin;
a buffering agent; and
a salt, wherein:
the dry powder insulin formulation comprises between about 70 and 95% w/w of insulin, between about 5 and 30% w/w of the buffering agent, and less than about 1% w/w of the salt; and
the dry powder insulin formulation has less than about 5% water content.

2. The dry powder insulin formulation for reconstitution of claim 1, wherein:
the buffering agent comprises one or more of citrate, phosphate, acetate, Tris(hydroxymethyl)aminomethane (TRIS), glycine, or glycylglycine.

3. The dry powder insulin formulation for reconstitution of claim 1, wherein:
a feedstock used to produce the dry power insulin formulation has a pH of between about 7.5 and 9.0 and comprises less than about 0.6% w/w zinc.

4. The dry powder insulin formulation for reconstitution of claim 1, wherein:
the dry powder insulin formulation is spray-dried.

5. The dry powder insulin formulation for reconstitution of claim 1, wherein:
the dry powder insulin formulation is lyophilized.

6. The dry powder insulin formulation for reconstitution of claim 1, wherein:
the dry powder insulin formulation does not contain a stabilizer or a preservative.

7. A method of reconstituting a dry powder insulin formulation, the method comprising:
providing a dry powder insulin formulation comprising:
insulin;
a buffering agent; and
a salt, wherein the dry powder insulin formulation has less than about 5% water content; and
combining the dry powder insulin formulation with a solvent to reconstitute the dry powder insulin formulation into a liquid insulin formulation having an insulin concentration between about 30 IU (1 mg/mL) and 2000 IU (70 mg/mL).

8. The method of reconstituting a dry powder insulin formulation of claim 7, wherein:
the liquid insulin formulation has a concentration of greater than about 35 mg/mL.

9. The method of reconstituting a dry powder insulin formulation of claim 7, wherein:
the solvent is water.

10. The method of reconstituting a dry powder insulin formulation of claim 7, wherein:
combining the dry powder insulin formulation with the solvent reconstitutes the dry powder insulin formulation in between about 60 and 120 seconds.

11. The method of reconstituting a dry powder insulin formulation of claim 7, wherein:
the dry powder insulin formulation does not contain a stabilizer or a preservative.

12. A method of reconstituting a dry powder insulin formulation, the method comprising:
providing a dry powder insulin formulation in a first chamber of a syringe, the dry powder insulin formulation comprising:
insulin;
a buffering agent; and
a salt, wherein the dry powder insulin formulation has less than about 5% water content;
providing a solvent in a second chamber of the syringe;
introducing the solvent into the first chamber to combine the dry powder insulin formulation with the solvent to reconstitute the dry powder insulin formulation into a liquid insulin formulation;
separating the second chamber from the first chamber; and
transferring the liquid insulin formulation into an insulin delivery device.

13. The method of reconstituting a dry powder insulin formulation of claim 12, wherein:
the delivery device comprises a pen injector, an inhaler, or a liquid nebulizer.

14. The method of reconstituting a dry powder insulin formulation of claim 12, wherein:
the dry powder insulin formulation does not contain a stabilizer or a preservative.

15. The method of reconstituting a dry powder insulin formulation of claim 12, wherein:
transferring the liquid insulin formulation to the insulin delivery device comprises inserting the first chamber into an insulin delivery device.

16. A method of manufacturing a dry powder insulin formulation for later reconstitution, comprising:
dissolving insulin into a feedstock having a pH of between about 7.5 to 9.0 to form an insulin-containing solution, the feedstock comprising a solvent, a buffering agent and a salt; and
drying the insulin-containing solution to form a dry powder insulin formulation comprising between about 70 and 95% w/w of insulin, between about 5 and 30% w/w of the buffering agent, less than about 1% w/w of the salt, and less than about 5% water content.

17. The method of manufacturing a dry powder insulin formulation for later reconstitution of claim 16, wherein:
drying the insulin-containing solution comprises spray drying the insulin-containing solution.

18. The method of manufacturing a dry powder insulin formulation for later reconstitution of claim 17, wherein:
an inlet temperature of gas used to spray dry the insulin-containing solution is between about 110° C. and 160° C.; and
an outlet temperature of gas used to spray dry the insulin-containing solution is between about 60° C. and 95° C.

19. The method of manufacturing a dry powder insulin formulation for later reconstitution of claim 16, wherein:
drying the insulin-containing solution comprises lyophilizing the insulin-containing solution.

20. The method of manufacturing a dry powder insulin formulation for later reconstitution of claim 16, wherein:
dry particles of the dry powder insulin formulation have a mass median diameter (MMD) of less than about 2 to 50 μm.
